SEQUENCE LISTING (13 sequences)

SEQ ID NO: 1 - DNA, 603 bp, Artificial Sequence
  Fully synthetic
atggccactgaagcaccgatagaggcgacggaggttccgccggcgtcagcgacggagacggtggcgaagcagccacataagctagagaggagatggacattctggttcgataatcaatctaagccgaaacaaggagccgcttggggaagttctcttcgaaaagcttatactttcgaaactgttgaggaattctggagtttatatgatcagatattcaagcccagcaagttgactgctaatgcggactttcatttgttcaaagctgggattgagcccaaatgggaagatcctgagtgtgctagtggtggcaagtggactgttacgagcagcagaaaggctaatcttgagactatgtggcttgaaactctgatggcattggtcggtgagcagtttgatgagtcagaggagatatgtggagtggttgccagtgtacgtcggagtcaggataaactttccttatggactaagactgcctccaatgaagcaattcaggtgagcattggtaggaagtggaaggagatcattgatgctgaaaaaatatcctatagtttccatgatgactctaaaagggaaaggtcagctaagagtcgatatactgtgtga

SEQ ID NO: 2 - protein, 200 aa, Artificial Sequence
  Fully synthetic
MATEAPIEATEVPPASATETVAKQPHKLERRWTFWFDNQSKPKQGAAWGSSLRKAYTFETVEEFWSLYDQIFKPSKLTANADFHLFKAGIEPKWEDPECASGGKWTVTSSRKANLETMWLETLMALVGEQFDESEEICGVVASVRRSQDKLSLWTKTASNEAIQVSIGRKWKEIIDAEKISYSFHDDSKRERSAKSRYTV

SEQ ID NO: 3 - DNA, 24 bp, Artificial Sequence
  Fully synthetic
atggccactgaagcaccgatagag

SEQ ID NO: 4 - DNA, 20 bp, Artificial Sequence
  Fully synthetic
tcacacagtatatcgactct

SEQ ID NO: 5 - DNA, 24 bp, Artificial Sequence
  Fully synthetic
ggcacgcctctatcggtgcttcag

SEQ ID NO: 6 - DNA, 24 bp, Artificial Sequence
  Fully synthetic
aaacctgaagcaccgatagaggcg

SEQ ID NO: 7 - DNA, 25 bp, Artificial Sequence
  Fully synthetic
ggcactagagaggagatggacattc

SEQ ID NO: 8 - DNA, 25 bp, Artificial Sequence
  Fully synthetic
aaacgaatgtccatctcctctctag

SEQ ID NO: 9 - DNA, 25 bp, Artificial Sequence
  Fully synthetic
aaggaatctttaaacatacgaacag

SEQ ID NO: 10 - DNA, 27 bp, Artificial Sequence
  Fully synthetic
caattccattacgcctctccgttcgct

SEQ ID NO: 11 - DNA, 28 bp, Artificial Sequence
  Fully synthetic
ggaacaaaatccgaatttatcaataact

SEQ ID NO: 12 - DNA, 21 bp, Artificial Sequence
  Fully synthetic
aactcaagagtcgttggaaca

SEQ ID NO: 13 - DNA, 22 bp, Artificial Sequence
  Fully synthetic
caagcaagcatatacacttagc

(19) United States
(12) Patent Application Publication
(10) Pub. No.: US 2021/0189419 A1
(43) Pub. Date: Jun. 24, 2021

(54) LOSS-OF-FUNCTION GENE OF DOMINANT GENE EIFISO4E-S RESISTANT TO TVBMV AND USES THEREOF

(71) Applicant: Yunnan Academy of Tobacco Agricultural Sciences, Kunming (CN)

(72) Inventors: Yong LIU, Kunming (CN); Changjun HUANG, Kunming (CN)

(21) Appl. No.: 17/191,643

(22) Filed: Mar. 3, 2021

Related U.S. Application Data
(63) Continuation-in-part of application No. PCT/CN2019/101922, filed on Aug. 22, 2019.

(30) Foreign Application Priority Data
Sep. 16, 2018 (CN) 201811077929.9

(51) Int. Cl. C12N 15/82; C07K 14/415

(52) U.S. Cl. CPC C12N 15/8283; C07K 14/415

(57) ABSTRACT

The disclosure provides a loss-of-function gene of eIFiso4E-S. The loss-of-function gene is resistant to tobacco vein banding mosaic virus (TVBMV) and represented by the sequence of SEQ ID NO: 1.

### CROSS-REFERENCE TO RELATED APPLICATIONS

This application is a continuation-in-part of International Patent Application No. PCT/CN2019/101922 with an international filing date of Aug. 22, 2019, designating the United States, now pending, and further claims foreign priority benefits to Chinese Patent Application No. 201811077929.9 filed Sep. 16, 2018. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of biotechnology in tobacco breeding, and more particularly, to a loss-of-function gene of a dominant gene eIFiso4E-S, an eIFiso4E-S knockout mutant and uses thereof for producing a tobacco resistant to Tobacco vein banding mosaic virus (TVBMV).

TVBMV is a typical member of viruses in the genus Potyvirus Y (also known as PVY). TVBMV affects crops of the family solanaceous, such as potato, tobacco, tomato and pepper, and has become a major disease of tobacco. TVBMV is non-persistent transmitted in the field by aphids. Insecticides are inefficient in controlling aphids that have rapid development and high fecundity in all life stages.

Tobacco is an important economic crop. Deletion of tobacco eIF4E1 gene (also abbreviated as eIF4E-1, with GenBank sequence accession number KF155696) can create a PVY-resistant tobacco. At present, there are few researches on the identification of TVBMV-resistance resources, breeding for disease resistance, as well as pathogen detection and identification related to tobacco.

### SUMMARY

A first object of the disclosure is to provide a loss-of-function of eIFiso4E-S. eIFiso4E-S is a dominant gene, and the produced loss-of-function gene is resistant to TVBMV.

A second object of the disclosure is to provide an amino acid sequence of the loss-of-function of the dominant gene eIFiso4E-S.

A third object of the disclosure is to provide uses of the loss-of-function of the dominant gene eIFiso4E-S, particularly, to provide uses of eIFiso4E knockout) (eifiso4e-s^{KO} in breeding of a tobacco variety resistant to TVBMV.

A fourth object of the disclosure is to provide a tobacco variety, seeds, vegetative propagules thereof comprising the gene eifiso4e-s^{KO}.

A fifth object of the disclosure is to provide an eIFiso4E-S knockout kit.

In one aspect, the disclosure provides a loss-of-function gene of a dominant gene eIFiso4E-S, the loss-of-function gene being resistant to tobacco vein banding mosaic virus (TVBMV) and the dominant gene eIFiso4E-S is represented by a sequence of SEQ ID NO: 1.

In another aspect, the disclosure provides a polypeptide being coded by the dominant gene, and the polypeptide represented by a sequence of SEQ ID NO: 2.

The disclosure also provides a method of preparing a tobacco variety comprising eifiso4e-s^{KO} and being resistant to TVBMV, the method comprising knocking out gene eIFiso4E-S)(eifiso4e-s^{KO} of a target tobacco through chromosome segment substitution, gene introduction, gene editing, gene silencing, or physical and chemical mutagenesis.

In a class of this embodiment, chromosome segment substitution comprises introducing a chromosome segment comprising eifiso4e-s^{KO} into a target tobacco through hybrid breeding and protoplast fusion, thereby obtaining the tobacco variety resistant to TVBMV.

In a class of this embodiment, gene introduction comprises introducing an exogenous gene eifiso4e-s^{KO} into the target tobacco, thereby obtaining the tobacco variety resistant to TVBMV.

In a class of this embodiment, gene editing comprises inserting, deleting, or modifying a specific nucleotide base of the dominant gene eIFiso4E-S in the target tobacco, thereby acquiring the loss-of-function gene of the sequence shown in SEQ ID NO: 1 and obtaining the tobacco variety resistant to TVBMV.

In a class of this embodiment, physical and chemical mutagenesis comprises using a physical and chemical mutagen to destroy the function of the sequence shown in SEQ ID NO: 1 of the target tobacco, thereby obtaining the tobacco variety resistant to TVBMV.

In a class of this embodiment, the target tobacco is a tobacco carrying a recessive resistance gene va or an eIF4E1 knockout (eif4E1^{KO}) tobacco.

In a class of this embodiment, the tobacco variety comprising eifiso4e-s^{KO} and being resistant to TVBMV comprises a tobacco plant, a seed, and a vegetative propagule thereof.

The disclosure also provides a Knockout kit comprising the loss-of-function gene of the dominant gene eIFiso4E-S.

The loss-of-function of the dominant gene eIFiso4E-S may be used to prepare an eifiso4e-s knockout)(eifiso4e-s^{KO} material through gene editing, physical and chemical mutagenesis, germplasm resource screening, artificial gene synthesis, gene expression by interference, etc. The eifiso4e-s^{KO} material may be inserted into an eIF4E1 knockout (eif4e1^{KO}) material or an eIF4E1 loss-of-function (eif4e1^{LOF}) material, thereby obtaining different tobacco varieties including but not limited to eifiso4e-s^{KO} eif4e1^{KO}, eifiso4e-s^{LOF} eif4e1^{KO}, eifiso4e-s^{KO} eif4e1^{LOF}, and eifiso4e-s^{LOF}eif4e1^{LOF}. The tobacco varieties have resistance to TVBMV.

The disclosure extracts a loss-of-function gene of the dominant gene eIFiso4E-S having a base sequence of SEQ ID NO: 1. SEQ ID NO: 1 may be obtained by the following methods: (1) searching genomic databases; (2) searching the tobacco genomic library or cDNA library with the sequence of SEQ ID NO: 1 as a probe; (3) designing and synthesizing a set of oligonucleotide primers according to the base sequence of SEQ ID NO: 1, and amplifying the loss-of-function gene of the dominant gene eIFiso4E-S from tobacco genome or cDNA by PCR with the set of oligonucleotide primers; (4) obtaining the loss-of-function gene of the dominant gene eIFiso4E-S by chemical synthesis; and (5) deleting at least one codon coding for amino acid residues, and/or mutating at least one base pair.

A knockout mutant for the gene having the sequence of SEQ ID NO: 1 and a mutant that cannot interact with the VPg of TVBMV can be obtained by the following methods:

(1) Gene editing techniques such as CRISPR-Cas9, TALEN, zinc finger nucleases. (2) Chemical mutagenesis involves use of chemical mutagen, such as ethyl methanesulfonate (EMS) and nitrite. (3) Physical mutagenesis involves use of radiation mutagenesis, such as gamma rays, X-rays, fast neutrons, and heavy ions. (4) A natural variant has a sequence similar to the gene that shown in SEQ ID NO: 1, but the function of the natural gene has been destroyed. There are at least one deletion and/or addition and/or substitution in at least one position in the natural variant compared with the sequence of SEQ ID NO: 1, so that the VPg of TVBMV cannot interact with the similar sequence, breaking the cycle of infection. The natural variant may be identified by well-known techniques used in molecular biology, such as polymerase chain reaction (PCR) and prior art hybridization techniques. The artificially generated variants also comprise synthetic polynucleotides, such as those generated by site directed mutagenesis while sharing sequence identity with the naturally generated sequences of the disclosure, and the variants are also resistance to TVBMV. In general, the artificial variants have a sequence with more than 90% identity with the sequence of SEQ ID NO: 1.

The polynucleotide variant can also be identified by comparing the sequence of SEQ ID NO: 2 with the amino acid sequence of the polypeptide encoded by the polynucleotide variant. The sequence identity between any two polypeptides is calculated using the sequence alignment programs and parameters. The sequence identity between any two polypeptides is greater than 90%.

The sequence identity between any two polypeptides may be calculated using molecular biological tools such as MEGA and BLAST.

The polypeptide encoded by the loss-of-function gene of the dominant gene eIFiso4E-S has an amino acid sequence of SEQ ID NO: 2 and is resistant to TVBMV.

Combination Methods:

An application method of the loss-of-function gene of the dominant gene eIFiso4E-S comprises: A) preparing an eIFiso4E-S knockout)(eifiso4e-s^{KO} material through gene editing, physical mutagenesis, chemical mutagenesis, germplasm resource screening, artificial gene synthesis, gene expression by interference, etc.; B) preparing an eIF4E1 knockout (eif4e1^{KO}) material using the same methods in A); and C) recombining the eifiso4e-s^{KO} material with the eif4e1^{KO} material to produce a recombinant material comprising eifiso4e-s^{KO} and eif4e1^{KO}.

A preferred application method comprises: (1) preparing an eIFiso4E-S knockout (eifiso4e-s^{KO}) material according to the methods in A), and preparing an eIF4E1 knockout (eif4e1^{KO}) material using the eifiso4e-s^{KO} material; or preparing an eif4e1^{KO} material according to the methods in A), and preparing an eifiso4e-s^{KO} material using the eif4e1^{KO} material.

A more preferred application method comprises: (1) knocking out elF4E1 gene of a tobacco plant; modifying a specific gene site of the tobacco through gene editing, chemical or physical mutagenesis; thereby obtaining a new tobacco comprising eifiso4e-s^{KO} eif4e1^{KO}; (2) knocking out eIF4E1 gene of a tobacco plant, determining a specific amino acid of eIFiso4E-S gene that interacts with a specific amino acid of the VPg of TVBMV; converting the specific amino acid of gene eIFiso4E-S to an amino acid that cannot interact with the specific amino acid of the VPg of TVBMV; thereby obtaining a tobacco plant comprising eifiso4e-s^{KO} eif4e1^{KO}; (3) knocking out eIF4E1 gene of a tobacco plant, screening a germplasm resource comprising eifiso4e-s^{KO} gene from a tobacco population; the eifiso4e-s^{KO} germplasm resource comprises wild and cultivated species of tobacco, as well as hybrid species of wild and cultivated species; crossing or backcrossing the eifiso4e-s^{KO} germplasm resource with the eif4e1^{KO} tobacco to obtain a tobacco plant comprising eifiso4e-s^{KO} eif4e1^{KO}; (4) when the gene eIF4E1 has normal function in a tobacco plant, modifying a specific site of the tobacco plant through gene editing, chemical mutagenesis, or physical mutagenesis; thereby obtaining a tobacco plant comprising eifiso4e-s^{KO}; crossing or backcrossing the eifiso4e-s^{KO} tobacco plant and an eif4e1^{KO} tobacco; thereby obtaining a tobacco plant comprising eifiso4e-s^{KO} eif4e1^{KO}; (5) when the gene eIF4E1 has normal function in a tobacco plant, modifying a specific amino acid of gene eIFiso4E-S by gene editing or mutant screening to an amino acid that cannot interact with the specific amino acid of the VPg of TVBMV; crossing or backcrossing the eifiso4e-s^{KO} tobacco plant and an eif4e1^{KO} tobacco; thereby obtaining a tobacco plant comprising eifiso4e-s^{KO} eif4e1^{KO}. The tobacco plant comprising gene eifiso4e-s^{KO} eif4e1^{KO} or gene eifiso4e-s^{KO} was used to screen a tobacco variety resistant to TVBMV.

The exogenous gene eifiso4e-s^{KO} are introduced into the target tobacco via transgenic methods or direct introduction. Agrobacterium transformation is the most commonly used transgenic method for plant genetic engineering. The methods for directly introducing a gene into either cells or tissues comprise microinjection, pollen tube passage method, electrical conductivity, gene gun, etc. The tissues differentiate and regenerate a whole transgenic tobacco.

The gene eIFiso4E-S was edited and its polypeptide cannot interact with the VPg of the TVBMV. Consequently, a material comprises the edited gene is resistant to TVBMV.

TVBMV interacts with two genes of tobacco eIF4E family to complete the cycle of infection. The two genes may be knocked out simultaneously, thereby having resistance to TVBMV. A preferred method for knocking out the two genes comprising: knocking out another gene in a common eIF4E1 knockout tobacco (va tobacco); thereby obtaining a breeding material resistant to TVBMV. For instance, the eifiso4e-s^{KO}/eif4e1 or eifiso4e-s^{KO}/va double mutant tobacco may be cultivated to obtain a gene resource or disease-resistant material which is resistant to TVBMV.

The disclosure provides tobacco varieties resistant to TVBMV, as well as seeds and vegetative propagules thereof. Further, the disclosure also provides a plurality of genetic engineering products including A: an eIFiso4E-S knockout kit, a transgenic cell line and a recombinant bacteria thereof; B: an eIF4E1 knockout kit, a transgenic cell line and a recombinant bacteria thereof; C: A combination of A and B; and D: an eIFiso4E-S and eIF4E1 knockout kit, a transgenic cell line and a recombinant bacteria thereof. The tobacco treated with anyone of the plurality of genetic engineering products have resistance to TVBMV.

### DETAILED DESCRIPTION

Unless otherwise specified in embodiments, methods refer to the conventional methods, and test materials are available from the commercial provider.

Tobacco materials comprises Nicotiana tabacum cv Yunyan87 (also known as Yunyan 87 which has a genotype of eIFiso4E-S/eIF4E1 that is susceptible to a TVBMV infection), Honghua Dajinyuan (which has a genotype of eIFiso4E-S/eIF4E1 that is susceptible to the TVBMV infection). 2-1398 (which has a genotype of eIFiso4E-S/va that is resistant to PVY infection). The tobacco materials and TVBMV are provided by Yunnan Academy of Tobacco Agriculture.

Total RNA was extracted from tobacco leaves using TRIzol reagent (Invitrogen; Carlsbad, Calif.) according to the manufacturer's instruction. Plasmid DNA purification kit, agarose gel DNA extraction kit, and DNA fragment purification kit were purchased from QIAGEN. Escherichia coli DH5a, Restriction endonucleases, DNA Marker, PrimeSTAR GXL DNA Polymerase, T4 DNA polymerase, T4 DNA ligase, and spectinomycin were purchased from Takara Bio Inc. and Roche Biotech Company. TRIzol plus RNA extraction kit was purchased from Invitrogen Corporation. The Escherichia coli DH5α, Agrobacterium tumefaciens stains EHA105 and C58C1 were stored for later use. pMD18-T vector was purchased from Takara Bio Inc.

### Example 1

Analysis of eIFiso4E Gene Sequence of Common Tobacco.

Pepper eIFiso4E gene (Genebank: DQ022080) was used as a reference and two homologous sequences of eIFiso4E gene in common tobacco (N. tabacum) were found in Genbank database by Blastn. The nucleotide sequences of eIFiso4E gene in Nicotiana sylvestris (N. sylvestris) and Nicotiana tomentosiformis (N. tomentosiformis) respectively have a high coincidence rate with that of common tobacco. The two nucleotide sequences were named eIFiso4E-S and eIFiso4E-T, respectively.

Specific primers for synthesizing the dominant gene eIFiso4E-S:

isoS-F:
(SEQ ID NO: 3)
(5′-ATGGCCACTGAAGCACCGATAGAG-3′)
isoS-R:
(SEQ ID NO: 4)
(5′-TCACACAGTATATCGACTCT-3′)

### Example 2

Cloning and Sequence Analysis of eIFiso4E Gene of Common Tobacco.

(1) Total RNA extraction from tobacco: Fresh young leaves were collected from common tobacco Honghua Dajinyuan and total RNA was extracted from the fresh young leaves using TRIzol reagent (Invitrogen). Total RNA was reverse transcribed into cDNA by RT-PCR using Oligo dT-Adapter as a primer.

(2) Cloning of eIFiso4E-S gene: cDNA was used as a template and eIFiso4E-S gene was amplified by PCR using the specific primers isoS-F and isoS-R. Total reaction volume for PCR was 50 μL, including 4.0 μL of 100 ng/μL cDNA, 10.0 μL of 5×PCR buffer, 4 μL of dNTPs (2.5 mmol/L each), 2.0 μL of the specific primer isoS-F, 2.0 μL of the specific primer isoS-R, 1 μL of PrimeSTAR GXL DNA Polymerase, and 27 μL of ddH₂O. All reagents were purchased from Takara. PCR conditions: 98° C. for 2 min, then 35 cycles of 98° C. for 10 s, 60° C. for 15 s, 68° C. for 1 min, followed by 68° C. for 10 min.

(3) Recovery and purification of PCR products: PCR products were electrophoresed on a 1.5% agarose gel with 1×TAE as the electrophoresis buffer. Bromophenol blue dye migrated at 120 V for 60 min. The gel was then transferred into a gel imaging system for recording the dye bands. The small portion containing DNA fragment of interest was cut out from the gel under UV light. The DNA fragment of interest was recovered with a DNA fragment purification kit (purchased from QIAGEN).

(4) Cloning and sequencing of PCR products: The recovered DNA fragment of interest was introduced into a cloning vector. 30 positive clones were screened, sequenced by Thermo Fisher Scientific Inc. (Guangzhou), and aligned to the eIFiso4E-S gene sequence. The nucleotide sequences showing more than 99% sequence identity were identified as the DNA sequence of the eIFiso4E-S gene. The eIFiso4E-S gene sequence was shown in SEQ ID NO: 1.

### Example 3

Polypeptide Sequence Encoded by eIFiso4E-S Gene.

The nucleotide sequence of eIFiso4E-S gene was translated to an amino acid sequence by a Molecular Evolutionary Genetics Analysis software MEGA. The amino acid sequence, also called a polypeptide sequence, was shown in SEQ ID NO: 2.

### Example 4

Construction of eIFiso4E-S Targeting Vector.

An eIFiso4E-S targeting vector was constructed using a plasmid pRGEB31 which was disclosed in the reference “Xie, K. and Y. Yang (2013). “RNA-guided gene editing in plants using a CRISPR-Cas system.” Mol Plant 6(6)”. In accordance with the principle of CRISPR/Cas9 technology, a target site was designed in the first exon of eIFiso4E-S gene. A PAM site was found on the sequence of the first exon (The PAM site is NGG or CCN which takes the form of

5′-NNNNNNNNNNNNNNNNNNNNNGG -3′
or
5′-CCNNNNNNNNNNNNNNNNNNN-3′).

Synthesis and annealing of gDNA oligo: the target site of gRNA was used as a template. Two target sites were determined for the first exon of eIFiso4E-S gene. The specific primers were as follows:

gRNA1F
(SEQ ID NO: 5)
5′-GGCAcgcctctatcggtgatcag-3′;
gRNA1R
(SEQ ID NO: 6)
5′-AAACctgaagcaccgatagaggcg-3′;
gRNA2F
(SEQ ID NO: 7)
5′-GGCActagagaggagatggacattc-3′;
gRNA2R
(SEQ ID NO: 8)
5′-AAACgaatgtccatctcctctctag-3′.

Primer annealing: a set of complementary DNA oligo was synthesized and annealed to form a dsDNA.

An annealing system was as follows:

| | |
|---|---|
| F-primer: | 20 ul |
| R-Primer: | 20 ul |
| 10X Annealing buffer: | 5 ul |
| H₂O: | 5 ul |

The annealing conditions: 95° C. for 5 min, 90° C. for 1 mim, 80° C. for 1 mim, 70° C. for 1 mim, 60° C. for 1 mim, 50° C. for 1 mim, 40° C. for 1 mim, 30° C. for 1 mim, 20° C. for 1 mim, and 10° C. for 1 mim.

A Restriction Digestion System of pRGEB31 Plasmid:

| | |
|---|---|
| Plasmid: | 5 ul |
| 10X buffer: | 5 ul |
| BsaI: | 2 ul |
| H₂O: | 37 ul |

The digested products of large fragments were recovered:

A System for Connecting the Recovered Products and the dsDNA.

| | |
|---|---|
| Recovered products | 3 ul |
| dsDNA | 10 ul |
| T4 DNA buffer | 2 ul |
| T4 DNA Ligase | 1 ul |
| H₂O | 4 ul |

The connected product was transformed into Escherichia coli and the colonies were screened using colony PCR for positive colonies. The forward primer for PCR: OsU3 5′F 5′-aaggaatctttaaacatacgaacag-3′ (SEQ ID NO: 9). The reverse primer for PCR was the primer gRNA1R or gRNA2R. The positive colonies were incubated in a shaking incubator and sequenced to analysis whether gRNA was correct. The primer for sequencing was OsU3 5′F.

### Example 5

Plant Transformation and Detection of eIFiso4E-S Targeting Vector.

The recombinant plasmid pRGEB31 was transformed into Agrobacterium GV3101 by electroporation. Calluses were induced from the wild-type tobacco Yunyan 87 and the tobacco 2-1398 comprising a homozygous va gene locus, followed by Agrobacterium-mediated plant transformation. The induced calluses were infected with Agrobacterium GV3101 and screened for hygromycin B resistant calluses. The resistant calluses differentiated and regenerated a whole transgenic tobacco.

Detection of eIFiso4E-S Gene Mutation in Transgenic Tobacco.

Specific primers were designed upstream and downstream of the target site in accordance with eIFiso4E-S gene sequence. The specific primers were as follows:

EditestF:
(SEQ ID NO: 10)
5′ -caattccattacgcctctccgttcgct -3′;
EditestR:
(SEQ ID NO: 11)
5′-ggaacaaaatccgaatttatcaataact-3′.

The genomic DNA of the transgenic tobacco was used as a template, and the eIFiso4E-T gene was amplified by PCR using the specific primers EditestF and EditestR. The PCR products were sequenced by Thermo Fisher Scientific (Guangzhou) using the primer EditestF.

### Example 6

Recombination of eIFiso4E-S^{KO} and Va Leads to Resistance to TVBMV.

The eIFiso4E-S targeting vector in Example 4 was transformed into the tobacco 2-1398, and the resulting tobacco was named tobacco eifiso4e-s^{KO}/va. Genomic DNA was extracted from TO generation seedlings, amplified by PCR, sequenced, and screened for tobacco 2-1398g1-1C which comprises eIFiso4E-S^{KO}. Referring to SEQ ID NO: 1, a base A was added or deleted at position 11 in the sequence of eIFiso4E-S^{KO} compared with eIFiso4E. The tobacco 2-1398g1-1C were selfed, producing Ti generation seeds. Ti generation seedlings were nursed in a plot by conventional methods until each seedling has 4-5 leaves. Genomic DNA was extracted from the leaves, and screened for the individuals which are homozygous/biallelic for eIFiso4E-S gene and wild-type for eIFiso4E-T gene. Diseased leaves infected with TVBMV were squeezed into juice, diluted 40 times, and inoculated on the individuals. The va tobacco 2-1398 was used as a control group. The disease was investigated on the 14^{th}, 21^{st}, and 28^{th} after inoculation. Referring to Table 1, the control group (va tobacco 2-1398) had an incidence rate of 100% on 14^{th} after inoculation. The eifiso4e-s^{KO}/va tobacco had an incidence rate of 0, which shows a strong resistance to TVBMV.

TABLE 1
Resistance of tobacco eifiso4e-s^{KO}/va against TVBMV

| | | | Incidence rate, % | | | |
|---|---|---|---|---|---|---|
| Materials | Genotype | Number | 14 dpi | 21 dpi | 28 dpi | 35 dpi |
| 2-1398g1-1C | eifiso4e-s^{KO}/va | 24 | 0 | 0 | 0 | 0 |
| 2-1398 | va | 32 | 100 | 100 | 100 | 100 |

On the 32^{nd} day after inoculation, the leaves from each individual were collected for total RNA extraction. Oligo T primers were used in reverse transcription to obtain cDNA. PCR primers were designed according to the VPg sequence of TVBMV. PCR was carried out using an annealing temperature of 60° C. The amplified product was 765 bp in size.

TVBMVVPg_F:
(SEQ ID NO: 12)
5′ -AACTCAAGAGTCGTTGGAACA-3′;
TVBMVVPgR:
(SEQ ID NO: 13)
5′ -CAAGCAAGCATATACACTTAGC-3′.

No target bands were amplified in the 12 samples which are collected from the tobacco 2-1398g1-1C inoculated with TVBMV, but target bands were amplified in the tobacco 2-1398 inoculated with TVBMV. The results further confirmed that the tobacco 2-1398g1-1C is resistant to TVBMV, while the control 2-1398 is sensitive to TVBMV.

### Example 7

Breeding Application of Allele eifiso4e-s^{KO} of eIFiso4E-S

An allele of the gene eIFiso4E-S having the sequence of SEQ ID NO: 1 was named eifiso4e-s^{KO}. A chromosome fragment containing the allele eifiso4e-s^{KO} was transferred into a target tobacco by conventional breeding methods. The target tobacco may be va or eif4E1^{KO} tobacco. Germplasm resources comprising eifiso4e-s^{KO} were screened from a tobacco population by a functional or linked molecular marker of gene eifiso4e-s^{KO}, or by artificial inoculation of TVBMV. The germplasm resources comprise wild species of tobacco, hybrid and cultivated species of wild and cultivated species. The chromosome fragment comprising the gene eifiso4e-s^{KO} was screened from the germplasm resources and introduced into the target tobacco through conventional hybrid breeding, protoplast fusion, or chromosome segment substitution, thereby obtaining a non-transgenic tobacco with increased in TVBMV resistance. The non-transgenic tobacco was bred into a commercial variety through hybridization, backcrossing, etc., thereby improving the resistance of tobacco against TVBMV.

### Example 8

Breeding Application of Gene-Edited eIFiso4E-S Gene.

The gene eIFiso4E-S in the target tobacco was modified using biotechnology such as gene editing, losing the biological function of interacting with TVBMV VPg. The resulting tobacco can resist TVBMV. The target tobacco may be va or eif4E1^{KO} tobacco.

It will be obvious to those skilled in the art that changes and modifications may be made, and therefore, the aim in the appended claims is to cover all such changes and modifications.

What is claimed is:

1. A loss-of-function gene of a dominant gene eIFiso4E-S, the loss-of-function gene being resistant to tobacco vein banding mosaic virus (TVBMV) and the dominant gene eIFiso4E-S being represented by a sequence of SEQ ID NO: 1.

2. A polypeptide being coded by the loss-of-function gene of claim 1, and the polypeptide represented by a sequence of SEQ ID NO: 2.

3. A method of preparing a tobacco variety comprising eifiso4e-s^{KO} and being resistant to TVBMV, the method comprising knocking out the dominant gene eIFiso4E-S (eifiso4e-s^{KO}) of a target tobacco through chromosome segment substitution, gene introduction, gene editing, gene silencing, or physical and chemical mutagenesis.

4. The method of claim 3, wherein chromosome segment substitution comprises introducing a chromosome segment comprising eifiso4e-s^{KO} into the target tobacco through hybrid breeding and protoplast fusion, thereby obtaining the tobacco variety resistant to TVBMV.

5. The method of claim 3, wherein gene introduction comprises introducing an exogenous gene eifiso4e-s^{KO} into the target tobacco, thereby obtaining the tobacco variety resistant to TVBMV.

6. The method of claim 3, wherein gene editing comprises inserting, deleting, or modifying a specific nucleotide base of the dominant gene eIFiso4E-S in the target tobacco, thereby destroying the function of the sequence shown in SEQ ID NO: 1 and obtaining the tobacco variety resistant to TVBMV.

7. The method of claim 3, wherein physical and chemical mutagenesis comprises using a physical and chemical mutagen to destroy the function of the sequence shown in SEQ ID NO: 1 of the target tobacco, thereby obtaining the tobacco variety resistant to TVBMV.

8. The method of claim 3, wherein the target tobacco is a tobacco carrying a recessive resistance gene va or an eIF4E1 knockout (eif4E1^{KO}) tobacco.

9. The method of claim 3, wherein the tobacco variety comprises a tobacco plant, a seed, and a vegetative propagule thereof.

10. A Knockout kit comprising the loss-of-function gene of the dominant gene eIFiso4E-S of claim 1.
